(19) United States
(12) Patent Application Publication
(10) Pub. No.: US 2009/0317465 A1
(43) Pub. Date: Dec. 24, 2009

(54) COMPOSITION AND METHOD OF PREPARATION OF RELEASE SYSTEMS FOR CONSTANT (ZERO-ORDER) RELEASE OF ACTIVE AGENTS

(75) Inventor: Nicholas A. Peppas, Austin, TX (US)

Correspondence Address: CHALKER FLORES, LLP, 2711 LBJ FRWY, Suite 1036, DALLAS, TX, 75234, US

(73) Assignee: APPIAN LABS, LLC, Austin, TX (US)

(21) Appl. No.: 12/485,710

(22) Filed: Jun. 16, 2009

Related U.S. Application Data
(60) Provisional application No. 61/061,866, filed on Jun. 16, 2008.

(51) Int. Cl. A61K 9/24

(52) U.S. Cl. 424/472

(57) ABSTRACT

The present invention includes methods and compositions for pharmaceutical compositions that exhibit substantially constant release profile. The composition includes at least two layers with different drug to polymer ratios. The layers can be arranged in any order to achieve desirable pharmaceutical effects.

### CROSS-REFERENCE TO RELATED APPLICATIONS

This application claims priority to U.S. Provisional Application Ser. No. 61/061,866, filed Jun. 16, 2008, the entire contents of which are incorporated herein by reference.

### BACKGROUND OF THE INVENTION

Without limiting the scope of the invention, its background is described in connection with composition and methods to prepare a composition with various release profiles.

A major unsolved problem in the drug delivery field is the development of pharmaceutical formulations that can release drugs at prescribed delivery rates over a period of time. Currently, conventional pharmaceutical formulations (tablets, capsules, caplets, etc) do not provide a constant release of a drug over a period of time. As seen from standard pharmaceutical references (e.g., J. Robinson and VHL Lee, Controlled Drug Delivery, second edition, Dekker, New York, 1987), tablets produced by compression of microparticles do not provide conditions for constant release of drugs when taken orally, buccally, sublingually, ocularly, rectally or by other conventional routes of administration.

Conventional pharmaceutical formulations are prepared by incorporating various excipients and active agents (e.g., drugs) at different concentrations and compressing the ensuing mixture to achieve useful tablets and related systems. Such formulations are usually triggered by the physiological environment. For example, in oral delivery systems the pharmaceutical formulation is placed in contact with the gastrointestinal (GI) contents such as the presence of water, ions, etc. This triggering process naturally leads to a decrease of the drug delivery rate over time. This rate is defined as the amount (expressed in molar or mass quantities) of drug per surface area and per time of release.

Such pharmaceutical formulations and controlled release systems work due to the associated swelling in the administration route, the swelling being provided by physiological liquids naturally available in the mouth (saliva), stomach (gastrointestinal fluid), etc. Formulations for drug delivery are very often prepared as monoliths, tablets or matrices, formed by compression of hydrophilic microparticulate powders. These formulations are typically composed of a drug and a hydrophilic swellable excipient (polymer). The amount of polymer usually ranges from 10 to 30% of the total weight of the matrix. Many natural or synthetic polymers such as xanthan gum, guar gum, amylose starches, karaya gum, poly(ethylene oxide) (PEO), poly(vinyl alcohol) (PVA) and others have been used. Various cellulose derivatives such as hydroxypropyl methyl cellulose (HPMCs), hydroxypropyl cellulose (HPCs), carboxymethyl cellulose (CMC) and ethyl cellulose (EC) are certainly the most widely used class of polymers for their manufacture.

One such example can be found in the U.S. Pat. No. 5,780,057, which describes a pharmaceutical form for oral administration including a two- or three-layered tablet, with at least one layer that can rapidly swell by contact with biological and/or aqueous fluids, the swelling results in a considerable increase in the tablet volume. The patent shows a prolonged residence of the pharmaceutical form at the gastric level and therefore allows a slow release of the active ingredient from the pharmaceutical form to the stomach and/or the first tract of the intestine.

Another example can be found in the U.S. Pat. No. 5,681,583, which teaches multilayered controlled-release solid pharmaceutical composition in tablet form suitable for oral administration having at least two layers containing active material in association with excipients and additives. One layer of the tablet releases a portion of the drug quickly while the other layer and optionally further layers release portions of the drug more gradually. The patent also teaches systems for the release of active principles which are capable of releasing active principle(s) into an aqueous medium at a controlled rate. For example, a monolithic system for the controlled release of active principles is that that includes: at least one swelling layer containing one or more active principles, in a matrix of swellable, hydrophilic polymers; at least one erodible and/or soluble layer comprising excipients and/or water soluble polymers, possibly containing one or more active principles, either the same or different from those present in the layer, the erodible and/or soluble layer being in contact with the swelling layer(s).

Yet another example can be found in the U.S. Pat. No. 7,300,668, which discloses controlled release dosage forms and methods of designing and manufacturing dosage forms to obtain specific release profiles, for example, zero-order release profiles, escalating release profiles or decreasing release profiles. The dosage forms that Lewis teaches include spatial variation of API concentration in the dosage form and can include nested regions. Dosage forms may be manufactured by any appropriate method for obtaining the internal structure as disclosed herein for producing substantially zero-order release profiles. The patent further includes methods of manufacturing such dosage forms, such as by three-dimensional printing, possibly also including compression of the dosage form after three-dimensional printing. Lewis further includes methods of designing such dosage forms. Release profiles from non-uniform distributions of API concentration may be predicted based on simple experiments with uniform-concentration dosage forms.

Finally, United States Patent Application Publication Number 2007/0009599 teaches a tablet for the controlled release of a drug. The tablet is in the form of an asymmetrically coated tablet so that immediate release or time-delayed release times can be precisely controlled and the extended release slab may provide zero-order or first-order extended release and pulsatile release depending on the excipients used in the tablet formulations. The core of the tablet is coated with an asymmetrical coating, that is, a coating with regions having different properties. The coatings may include drugs in varying concentrations. Further, different regions of the coating may have different rates of dissolution. The core of the tablet may be provided with a constant cross-sectional area along a longitudinal length of the tablet, a coating having a first region with a more rapid rate of dissolution than a second region. The dissolution of the first region exposes only the cross-sectional area to the dissolution medium. The second region of the coating prevents any other portion of the core of the tablet from being exposed to the dissolution medium. Therefore, since the cross-sectional area remains constant as it is dissolved, the rate of release of the drug from the core of the tablet remains constant. The cross-sectional area may be of any geometrical configuration so long as the area remains constant as the core dissolves.

However, all of the above mentioned references do not posses a simple preparation method for formulating the composition, and most of them can not provide a constant release profile of the active agent. Accordingly, there exists a need in the industry for a pharmaceutical composition having a constant or zero-order release profile, with simple methods of preparing the pharmaceutical composition.

### SUMMARY OF THE INVENTION

The present invention relates to a novel pharmaceutical composition and methods of making the compositions that deliver one or more active agents with a substantially constant or zero order release profile. Described herein are designs, compositions and methods for the preparation of a composition that may be planar, cylindrical or of other geometric arrangements that include layered structures of coated or uncoated drug particles that may be compressed to produce tablets of multiple layers that deliver active agents or drugs with a substantially constant or zero order release profile. In some embodiments, the present invention may also be coated with one or more external layers to delay the release of drug for a desirable period of time. In addition, tablets can be prepared with intermediate drug-free layers, thus achieving one or more intermittent release at substantially constant rates followed by no release. The present invention can be applied to active agents, e.g., drugs, peptides, proteins, vaccines and other therapeutic agents, as well as cosmetic products, consumer products, food products and insecticides.

In one aspect, the present invention includes a single dose, pharmaceutical composition having a total of 2m−1 layers, with m being an integer between 2 and 100; a core having one or more active agents and one or more polymers with a mass ratio greater than about 1; at least one double layered first composition symmetrically positioned on top and bottom of the core, wherein the first composition contains one or more active agents and one or more polymers with a mass ratio between about 1 to about (n−1)/n; at least one double layered second composition symmetrically positioned on top and bottom of the core, wherein the second composition contains one or more active agents and one or more polymers with a mass ratio between about (n−1)/n to about (n−2)/n; and wherein n=m and is greater or equal to two.

In another aspect, the present invention can further include at least one double layered third composition symmetrically positioned on top and bottom of the core, wherein the third composition contain one or more active agents and one or more polymers with a mass ratio between about (n−2)/n to about (n−3)/n; wherein n=m and is greater or equal to three.

Yet in another aspect, the present invention can further include at least one double layered fourth composition symmetrically positioned on top and bottom of the core, wherein the fourth composition contain one or more active agents and one or more polymers with a mass ratio between about (n−3)/n to about (n−4)/n and wherein n=m and is greater or equal to four.

The pharmaceutical composition further comprises an external lateral coating having one or more substantially less water permeable or impermeable polymer layer covering the sides of the core to prevent dissolution or release of the one or more active agents.

Yet in another aspect, the present invention can further include at least one additional double layered composition having a mixture of the one or more active agents and the one or more polymers with a mass ratio of about 0.01-1.00 to about 0.01-1.00. In some aspects, the present invention includes at least one double layered composition having the one or more polymers with little to no active agent(s), and/or one or more layers of an extended-release coating. Furthermore, the present invention can include one or more inactive(s) or inactive agents and/or have a substantially constant release rate profile or a release profile in a zero order. In some embodiments, the present invention can include 1, 2, 3, 4, 5, 6, 7, 8, 9, 10, 20, 30, 40, 50, 60, 70, 80, 90, or 100 double layered compositions, and each double layer can be arranged in any order.

In one aspect, the present invention includes a method of making a multilayer composition by using the following steps: forming a core having one or more active agents and one or more polymers with a mass ratio greater than about 1; forming at least one double layered first composition symmetrically positioned on top and bottom of the core, wherein the first composition has one or more active agents and one or more polymers with a mass ratio between about 1 to about (n−1)/n; and forming at least one double layered second composition symmetrically positioned on top and bottom of the core, wherein the second composition has one or more active agents and one or more polymers with a mass ratio between about (n−1)/n to about (n−2)/n, wherein n is greater or equal to two.

In another aspect, the present invention further includes a method of making a multilayer composition by forming at least one double layered third composition symmetrically positioned on top and bottom of the core, wherein the third composition has one or more active agents and one or more polymers with a mass ratio between about (n−2)/n to about (n−3)/n, and wherein n is greater or equal to three. Yet in another aspect, the present invention further includes a method of making a multilayer composition by forming at least one double layered fourth composition symmetrically positioned on top and bottom of the core, wherein the fourth composition has one or more active agents and one or more polymers with a mass ratio between about (n−3)/n to about (n−4)/n, and wherein n is greater or equal to four.

In some aspects, the present invention further includes a method of making a multilayer composition by forming at least one addition layer of composition having a mixture of the one or more active agents and the one or more polymers having a molar ratio of about 0.01-1.00 to about 0.01-1.00.

In some aspects, the present invention further includes a method of making a multilayer composition by forming an external lateral coating having one or more substantially less water permeable or impermeable polymer layer covering the sides of the core to prevent dissolution or release of the one or more active agents. In some aspects, the present invention further includes a method of making a multilayer composition by forming at least one double layered composition comprising the one or more polymers.

In one aspect, the present invention further includes a method of making a multilayer composition by arranging each of the double layered composition in any order and/or forming one or more layers of an extended-release coating on the pharmaceutical composition. In some aspects, the present invention further includes a method of making a multilayer composition, which has a substantially constant release rate profile or a release profile in a zero order.

In one embodiment, the present invention includes a single dose, pharmaceutical composition having: a core containing one or more active agents and one or more polymers with a mass ratio greater than about 1; a first double layered composition symmetrically positioned on top and bottom of the core, wherein the first double layered composition has one or more active agents and one or more polymers with a mass ratio between about 1 to about 0.75; a second double layered composition symmetrically positioned on top and bottom of the core, wherein the second double layered composition has one or more active agents and one or more polymers with a mass ratio between about 0.75 to about 0.5; a third double layered composition symmetrically positioned on top and bottom of the core, wherein the third double layered composition has one or more active agents and one or more polymers with a mass ratio between about 0.5 to about 0.25; a fourth double layered composition symmetrically positioned on top and bottom of the core, wherein the fourth double layered composition has one or more active agents and one or more polymers with a mass ratio between about 0.25 to about 0.05; and an external lateral coating having one or more substantially less water permeable or impermeable polymer layer covering the sides of the core to prevent dissolution or release of the one or more active agents.

### BRIEF DESCRIPTION OF THE DRAWINGS

For a more complete understanding of the features and advantages of the present invention, reference is now made to the detailed description of the invention along with the accompanying figures and in which:

FIG. 1 is a schematic diagram of an embodiment of the present invention;

FIG. 2 is schematic diagram of another embodiment of the present invention;

FIG. 3 is a diagram of the release profile from the embodiment shown in FIG. 1;

FIG. 4 is a diagram of the release profile from the embodiment shown in FIG. 2; and

FIG. 5 is a graph showing the release profiles of the present invention.

FIG. 6 is a graph showing a release profile for acetaminophen having the table configuration shown on the right side of the figure.

FIG. 7 is a graph showing a release profile for acetaminophen having the table configuration shown on the right side of the figure.

FIG. 8 is a graph showing a release profile for theophylline having the table configuration shown on the right side of the figure.

FIG. 9 is a graph showing a release profile for acetaminophen having the table configuration shown on the right side of the figure.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Swelling tablets and matrix compositions are key systems in the field of drug delivery systems. Water swelling behavior into cross-linked polymers has been demonstrated over past several decades, and it becomes an important factor when considering how such compositions will behave in the GI tract, e.g., under what conditions they will give substantially constant rate release (zero order release) or continuously decreasing release (Korsmeyer, R. W., R. Gurny, E. Doelker, P. Buri, and N. A. Peppas, “Mechanisms of Solute Release from Porous Hydrophilic Polymers,” Intern. J. Pharm., 15 (1983) 25-35). Typically, zero order release is difficult or impossible to achieve with swellings systems. It requires that a carrier (polymer) be mixed with drug, dried for several hours or days, cut in disc shape and used as a composition. This is an unacceptable method for the pharmaceutical industry due to cost and time constrains.

The use of swellable materials for drug delivery applications has followed investigations of solvent and solute transport in polymeric systems, with several important observations and mathematical models developed which describe transport behavior in polymeric systems. For example, polymeric hydrogels have been used for the purpose of extended drug delivery, as well as drug targeting and patterned release profiles (Colombo, P., “Swelling-controlled Release in Hydrogel Matrices for Oral Route,” Adv. Drug Deliv. Rev., 11 (1993) 37-57.). Typical pharmaceutical compositions e.g., tablets, include an active ingredient compressed in a powder e.g., a cellulose derivative, and a disintegrant. However, these systems can only control drug release during the initial stages after the drug is placed in a body. Coated capsules can protect a drug for a period of time before releasing at an optimal site, thereby prolonging the active lifetime of the drug. These systems have limited applications for long-term drug delivery. Hydrogel delivery systems are capable of slow release of an imbedded drug, with release controlled by the rate of swelling and relaxation of the polymer (Klier, J. and N. A. Peppas, “Solute and Penetrant Diffusion in Swellable Polymers. VIII. Influence of the Swelling Interface Number on Solute Concentration Profiles and Release,” J. Control. Rel., 7 (1988) 61-68. Peppas, N. A. and A. R. Khare, “Preparation, Structure and Diffusional Behavior of Hydrogels in Controlled Release,” Adv. Drug Deliv. Rev., 11 (1993) 1-35).

Other examples, such as drug/solute release from pH-sensitive materials, can have additional factors influencing release profiles. Brannon-Peppas, L. and N. A. Peppas, “Solute and Penetrant Diffusion in Swellable Polymers. IX. The Mechanisms of Drug Release from pH-Sensitive Swelling-Controlled Systems,” J. Control. Rel., 8 (1989) 267-274) demonstrated swelling and release behavior of pH-sensitive hydrogels.

Pharmaceutical formulations based on swellable matrix tablets are typically activated by water, and drug release control depends on the interactions between water, polymer and drug. Water penetration into the matrix is the first step leading to polymer swelling and polymer and drug dissolution. The analysis of drug release from swellable matrix tablets often involves the treatment of release data with a power law as in equation 1. This can give information on drug release kinetics and on the mechanisms involved. The equation is based on a power law dependence of the fraction released on time. The exponent n, gets values that can range between 0.43 and 1, according to the geometry and the prevalence of the Fickian or the Case II (relaxation) transports. The equation has the following form:

$$\frac{{M}_{t}}{{M}_{∞}} = {kt}^{n} \qquad ( 1 )$$

where Mₜ is the drug released at time t, the M_{∞} is the amount of drug released at infinite time, k is a kinetics constant and n is the diffusional exponent.

Different types of swellable matrix tablets can be prepared by the use of hydrophilic polymers. The most common are the free swellable matrix tablets (polymer and solid drug mixed and compressed), in which swelling is unhindered. In order to introduce additional elements for drug release control, the swelling of these matrix tablets can be affected by matrix surface modification, as for instance by the application of partial coatings. Their function is to alter the swelling behavior and then the drug release. These modified matrix tablets are called swelling restricted matrix tablets. Other systems, in which swellable polymers are used as coating for delaying or controlling the diffusion of drug from the core, are the swelling-controlled reservoir systems.

The present invention uses novel and unique design to achieve substantially constant release of an active agent (e.g., drug, peptide, protein, oligonucleotides, active agent, bioactive agent, cosmetic product, consumer product, essential oil(s), insecticides, food products, or any combinations thereof). The present invention balances the quantity of material released with the path or distance traveled for release. This is achieved in a unique way with the present invention's design.

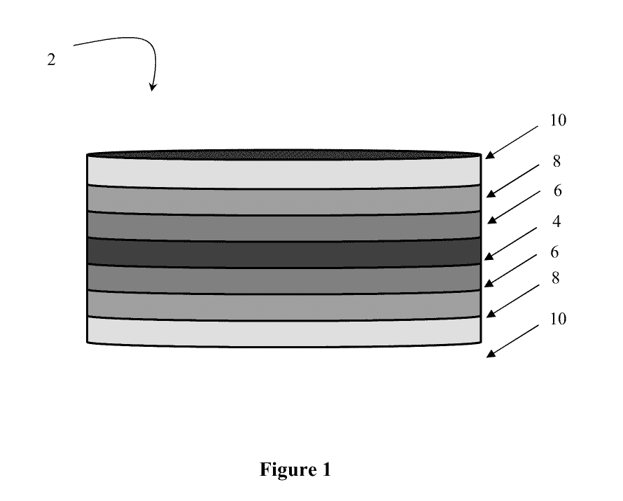

FIG. 1 shows a pharmaceutical composition 2 with at least 7 layers. The first layer 4 at has a mass ratio of drug (D) to polymer (P) of D/P of between about 0.75 and about 1.00, which corresponds to about 42.8 to about 50 wt % of drug. The first layer 4 of this composition can further include any drug or other active agent that can be granulated with pharmaceutical excipients such as hydroxy propyl methyl cellulose (HPMC), hydroxy propyl cellulose (HPC), poly(ethylene glycol) (PEG), poly(ethylene oxide (PEO), carboxymethylcellulose (CMC) and related compounds. The term polymer will be understood to include a “polymer mixture” that may include excipients with binders, stabilizers, lubricants and other pharmaceutical excipients typically used in pharmaceutical formulations. For example, a layer may include an “active agent” at a weight ratio (on a dry basis) with respect to the “polymer mixture” of great than 1.00.

Second layer 6 includes drug and polymer excipient at an initial mass ratio of drug (D) to polymer (P) of D/P of between about 0.50 and about 0.75, which corresponds to about 33.3 to about 42.8 wt % of drug.

Third layer 8 includes drug and polymer excipient at an initial mass ratio of drug (D) to polymer (P) of D/P of between about 0.25 and about 0.50, which corresponds to approximately about 20.0 to about 33.3 wt % of drug.

Finally, fourth layer 10 includes drug and polymer excipient at an initial mass ratio of drug (D) to polymer (P) of D/P of between about 0.05 or 0.01 and about 0.25, which corresponds to about 1.0 or 5.0 to about 20.0 wt % of drug. In this embodiment, these layers are compressed individually in a tablet press to produce a tablet with seven layers. In addition, small amounts of a lubricant (e.g., stearic acid, magnesium stearate) can also be added to achieve ease in tablet removal.

In FIG. 1, the drug distribution or drug content is higher in the center than at the outer regions of the tablet. Thus, when release (diffusion) takes place, the outer layers allow release first with a certain predesigned rate. Then, the inner layers allow release through an already swollen layer. But because they have higher drug quantity, they have longer to travel and release, they give the same overall rate. While there is some swelling on the polymeric matrix, in certain embodiments, the polymeric matrix remains relatively intact and drug is released through the polymeric matrix (albeit with some swelling) based on the concentration gradient between the tablet and the external environment.

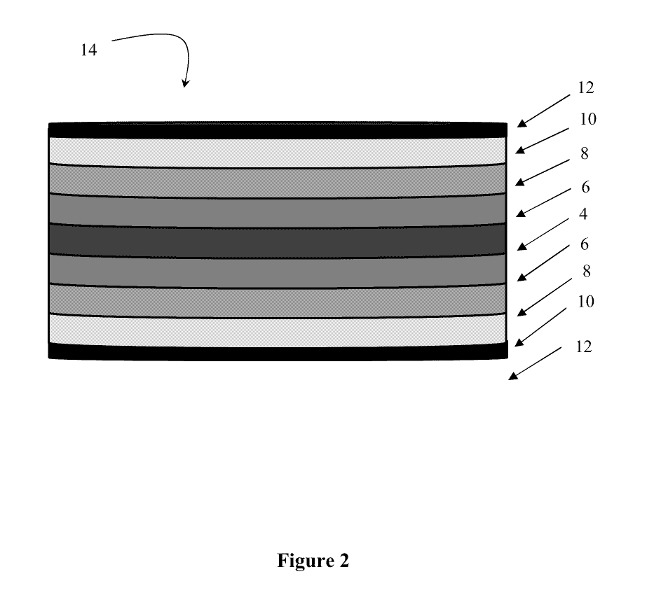

In certain embodiments, the various drug-containing layers shown in FIG. 1 can be separated by drug-free polymer excipient layers made of the same or different polymers. Thus, an intermittent release (delivery) can be achieve by observing consecutive substantially constant (zero-order) release periods separating by “silent” periods of non-release. Thus, a substantially constant and step-function release can be achieved. An example can be seen in FIG. 2, wherein the pharmaceutical composition 14 includes an extra layer 12 that has polymer only without any active agents.

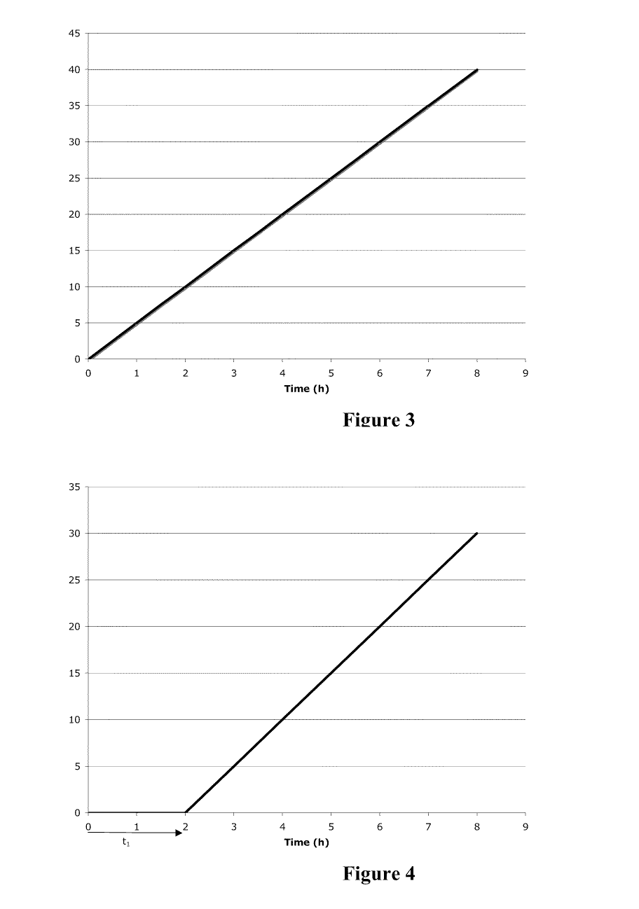

FIG. 3 shows a release profile of the embodiment shown in FIG. 1 with substantially constant drug delivery rates over time. The release rate can be adjusted by the ratio of drug to polymer (D/P) and by the molecular weight of the polymeric excipient used. High excipient molecular weights typically lead to slower release of the incorporated drug.

The overall composition may be adjusted to delay release by forming two external, drug-free layers made of the same polymer excipient, as shown in FIG. 2. Thus, an initial time lag, t₁, as shown in FIG. 4, with two external drug-free coatings can be achieved. This time lag can be positioned in any position alone the x-axis shown in FIG. 4, where a silence period with no active agent exists. This can be accomplished by having a pure polymer layer with no active agent. The time lag can be adjusted by using different types of polymer or a mixture of different polymers.

In certain embodiments, the one or more polymer used in the present invention is not bio-degradable during the release period of the composition, e.g., the polymer or mixture of polymers do not dissolve, or dissolve in a minute speed, in a physiological system during the ingested period. In a further embodiment, 3, 5, 9, 11, 13 and up to 50 such layers can be used for a total of about 500 mg pharmaceutical formulations.

In another embodiment of this composition, the two external coatings shown in FIG. 2 can be made of mucoadhesive polymer excipients such as Carbopol (poly(acrylic acid)), CMC, pectin, or any combinations thereof, thus rendering the overall device mucoadhesive for oral, buccal or sublingual applications. In certain embodiments, the present invention can be further manufactured as to release two or more different active agents (e.g., drug 1 in layers 4, 8, 12, etc., drug 2 in layers 6, 10, etc.) or a drug and a taste masking agent. In another embodiment, the same geometrical scheme can be used in different shapes, not only in cylindrical tablets (e.g., round, cube).

In certain embodiments, the present invention can include different layers arranged in any desirable order to produce desirable pharmaceutical effects. For example, the polymer only layer 12 depicted in FIG. 2 can be situated somewhere in the middle on the tablet, thereby providing a period of time where there are no release of any active agents. In addition, layers below the polymer only layer 12 can include a different active agent, thereby providing a subject with at least two different clinically essential drugs at different time periods without mixing the active agents in the subject's physiological system. This embodiment can further be expended so that the tablet taken can release a first active agent releasable in the stomach of a subject to treat stomach symptoms (disease), and a second active agent that is releasable in the intestine for different diseases or symptoms. Multiple modifications can further be accomplished by one skilled in the art to provide one drug or combination of drugs at different time periods and/or depending on the environment e.g., pH conditions, age of the patient. The examples mentioned so far and the figures all describe tablets with coating on the cylindrical face of the tablet where both ends are uncoated. The same performance can be achieved and follows the same principle for a tablet that has only one layer of each formulation composition (i.e., in FIG. 1 layers 4, 6, 8 and 10) where all surfaces of the tablet are coated by a substantially impermeable coating except one face, this uncoated face being the layer that is the outermost layer is the earlier descriptions. This tablet is then essentially one-half of the types of tablets described and depicted in FIGS. 1 and 2.

FIGS. 1 and 2 depict a 7 and 9 layers tablet, respectively. However, these are only shown as examples and further number of layers (e.g., 50 or 100 layers) can be accomplished by the skilled artisan. In some embodiments, each layer can also be made to include a gradient of polymer to drug ratio, thereby provided a finer control of release for the one or more active agents. For example, layer 10 shown in FIGS. 1 and 2 can have a gradient of polymer to drug ratio from top to bottom or from right to left depending on the final tablet geometry. This type of design would contribute a greater degree of control for a clinician for drugs that might have strong side effects to a subject (e.g., chemotherapeutic active agents).

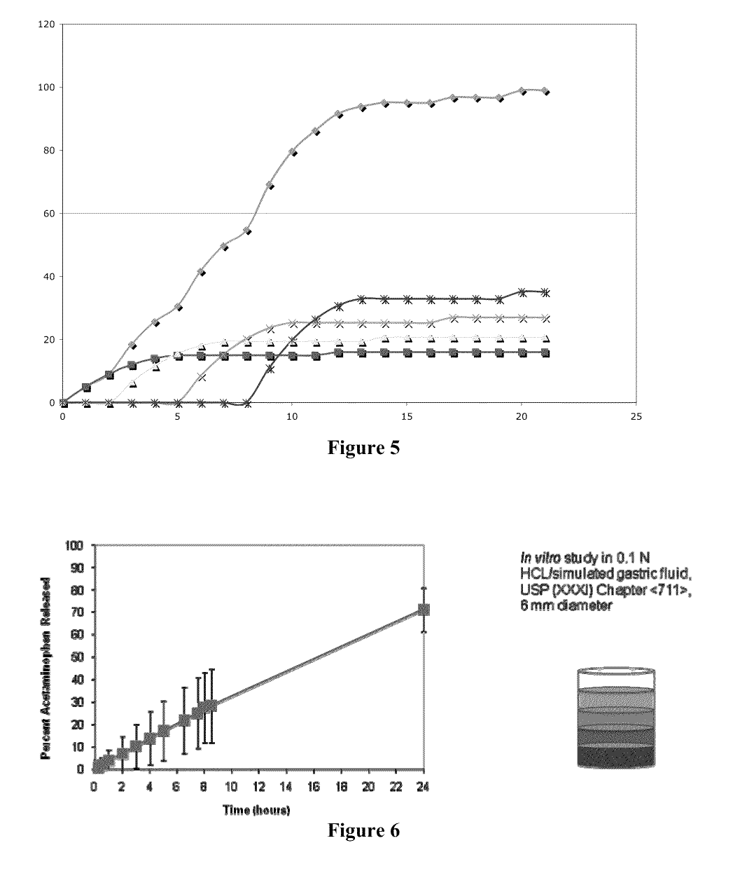

FIG. 5 is a graph showing the release profiles of the present invention. The y axis is amount released and thee x axis is time in number of hours. The line with the line with ⋄ depicts total drug released. The line with ▪ depicts the release of the outer layer. The line with A depicts the release of the second outer layer. The line with x depicts the release of the third outer layer. The line with * depicts the release of the inner layer.

FIG. 6 is a graph showing a release profile for acetaminophen having the table configuration shown on the right side of the figure and in which an in vitro study was conducted with release in 0.1N HCL/simulated gastric fluid, USP (XXXI) Chapter <711>, 6 mm diameter. The layers had the following characteristics: 5 layers, 45 mg drug per 125 mg tablet, 25 mg per layer, weight-percent (wt %) drug: 5, 15, 25, 50, 85.

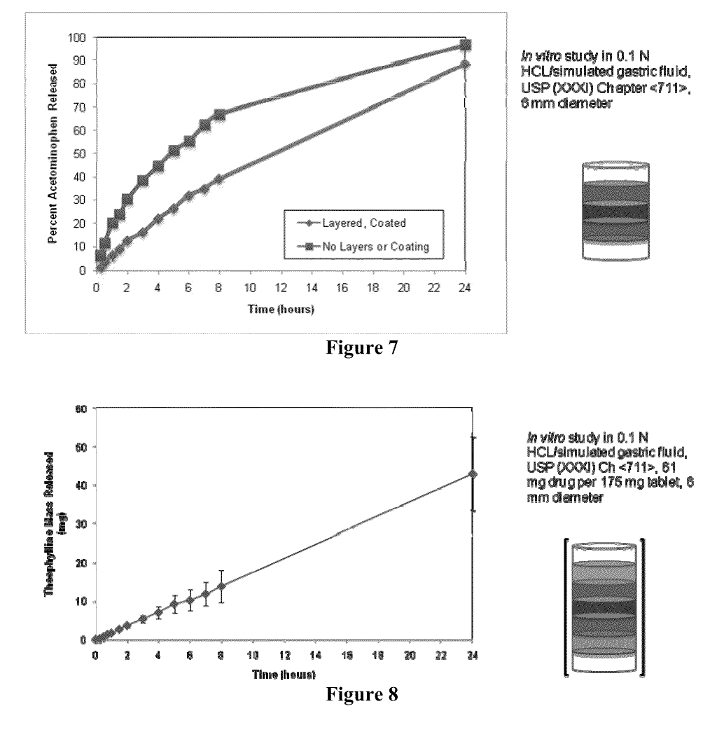

FIG. 7 is a graph showing a release profile for acetaminophen having the table configuration shown on the right side of the figure and in which an in vitro study was conducted with release in 0.1N HCL/simulated gastric fluid, USP (XXXI) Chapter <711>, 6 mm diameter. The layers had the following characteristics: Layered, Coated: 5 layers, 57 mg drug per 125 mg tablet, 25 mg per layer, wt % drug: 25, 50, 75, 50, 25; and No Layers or Coating: 1 layer uncoated, 55 mg drug per 122 mg tablet.

FIG. 8 is a graph showing a release profile for theophylline having the table configuration shown on the right side of the figure and in which an in vitro study was conducted in 0.1N HCL/simulated gastric fluid, USP (XXXI) Ch <711>, 61 mg drug per 175 mg tablet, 6 mm diameter. The layers had the following characteristics: A01-74, 7 layers, 25 mg per layer, wt % drug: 10, 25, 50, 75, 50, 25, 10.

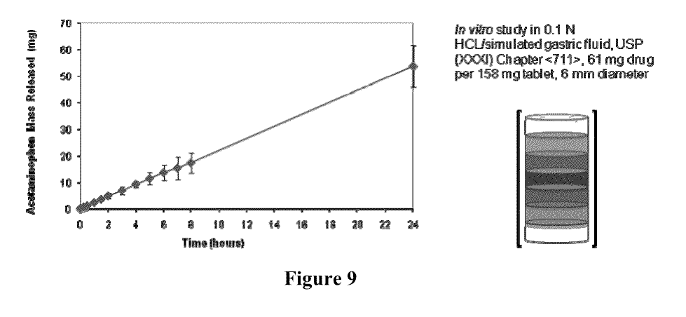

FIG. 9 is a graph showing a release profile for acetaminophen having the table configuration shown on the right side of the figure and in which an in vitro study was conducted with release in 0.1N HCL/simulated gastric fluid, USP (XXXI) Chapter <711>, 61 mg drug per 158 mg tablet, 6 mm diameter. The layers had the following characteristics: A03-19, 7 layers, 25 mg per layer, wt % drug: 10, 25, 50, 75, 50, 25, 10.

The term “zero order release” or “zero-order release rate” as used herein refers to a substantially constant, linear, continuous, sustained and controlled release rate of one or more active agents, i.e. the plot of mass of core released vs. time is linear.

As used herein, the mass ratio is defined as defined as total dry weight of the active agent(s) divided by total dry weight of the polymer(s).

As used herein, the n and m are integers from 1 to 100 e.g., 1, 2, 3, 4, 5, 6, 7, 8, 9, 10, 20, 30, 40, 40, 50, 60, 70, 80, 90, 100. The symbol m and n typically have the same value, but can be different depending on the embodiment. The equation (2 m−1) is typically used to show the total number of layers of the present invention, wherein equations (n−1)/n, (n−2)/n, (n−3)/n, (n−4)/n, (n−5)/n, (n−6)/n, (n−7)/n, (n−8)/n, (n−9)/n, (n−10)/n, (n−11)/n, (n−12)/n, (n−13)/n, (n−14)/n, (n−15)/n, (n−16)/n, (n−17)/n, (n−18)/n, (n−19)/n, (n−20)/n . . . and increments up to and including (n−100)/n are typically used to depict part the polymer to drug mass ratio.

As used herein, the terms “extended release” and “delayed release” as used herein is used to define a release profile to effect delivery of an active over an extended period of time. Extended release as used herein may also be defined as making the active ingredient available to the patient or subject regardless of uptake, as some actives may never be absorbed by the animal. Various extended release dosage forms may be designed readily by one of skill in art as disclosed herein to achieve delivery to both the small and large intestines, to only the small intestine, or to only the large intestine, depending upon the choice of coating materials, coating thickness and/or number of different layers with different polymer to drug ratio, and/or different manufacture process.

“Extended release” and “delayed release” compositions may be prepared and delivered so that release is accomplished at some generally predictable location in the lower intestinal tract more distal to that which would have been accomplished if there had been no delayed release alterations. A method for delay of release is, e.g., a coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention to achieve delivery to the lower gastrointestinal tract. Polymers and compatible mixtures thereof may be used to provide the coating for the delayed or the extended release of active ingredients, and some of their properties, include, but are not limited to: shellac, also called purified lac, a refined product obtained from the resinous secretion of an insect.

The term “enteric coating” as used herein relates to a mixture of pharmaceutically acceptable excipients that is applied to, combined with, mixed with or otherwise added to the carrier or composition. The coating may be applied to a compressed or molded or extruded tablet, a gelatin capsule, and/or pellets, beads, granules or particles of the carrier or composition. The coating may be applied through an aqueous dispersion or after dissolving in appropriate solvent. Additional additives and their levels, and selection of a primary coating material or materials will depend on the following properties: resistance to dissolution and disintegration in the stomach; impermeability to gastric fluids and drug/carrier/enzyme while in the stomach; ability to dissolve or disintegrate rapidly at the target intestine site; physical and chemical stability during storage; non-toxicity; easy application as a coating (substrate friendly); and economical practicality.

Colorants, detackifiers, surfactants, antifoaming agents, lubricants, stabilizers such as hydroxy propyl cellulose or methylated cellulose, acid/base can be added to the coatings of the present invention besides plasticizers to solubilize or disperse the coating material, and to improve coating performance and the coated product.

The pharmaceutically active agents useful in the practice of the present invention include, but are not limited to, nutraceuticals, vitamins, food additives, food supplements, antihistamines, decongestants, antitussives and/or expectorants. Other actives for use with the present invention include, but are not limited to: non-steroidal anti-inflammatory drugs (NSAIDs) and other analgesic drugs such as acetominophen and phenacetin. These materials are incorporated into the slow or controlled release compositions of the invention in amounts governed by the desired release characteristics of the material in such excipient base and such that conventional dosages comply with applicable FDA or other regulations.

Suitable excipients (active agents) are those used commonly to facilitate the processes involving the preparation of the solid carrier, the encapsulation coating, or the pharmaceutical dosage form. These processes include agglomeration, air suspension chilling, air suspension drying, balling, coacervation, comminution, compression, pelletization, cryopelletization, extrusion, granulation, homogenization, inclusion complexation, lyophilization, nanoencapsulation, melting, mixing, molding, pan coating, solvent dehydration, sonication, spheronization, spray chilling, spray congealing, spray drying, or other processes known in the art. The excipients may also be pre-coated or encapsulated, as are well known in the art.

Carriers: The carrier of the pharmaceutical compositions may be a powder or a multiparticulate, such as a granule, a pellet, a bead, a spherule, a beadlet, a microcapsule, a millisphere, a nanocapsule, a nanosphere, a microsphere, a platelet, a minitablet, a tablet or a capsule. A carrier may be a finely divided (milled, micronized, nanosized, precipitated) form of a matrix on which the active ingredient is disposed. Such matrix may be formed of various materials known in the art, such as, for example: sugars, such as lactose, sucrose or dextrose; polysaccharides, such as maltodextrin or dextrates; starches; cellulosics, such as microcrystalline cellulose or microcrystalline cellulose/sodium carboxymethyl cellulose; inorganics, such as dicalcium phosphate, hydroxyapitite, tricalcium phosphate, talc, or titania; and polyols, such as mannitol, xylitol, sorbitol or cyclodextrin. It should be emphasized that a substrate need not be a solid material, although often it will be a solid.

Other additives conventionally used in pharmaceutical compositions may be included in the present invention, which are known in the art. Such additives include, e.g.: anti-adherents (anti-sticking agents, glidants, flow promoters, lubricants) such as talc, magnesium stearate, fumed silica), micronized silica, polyethylene glycols, surfactants, waxes, stearic acid, stearic acid salts, stearic acid derivatives, starch, hydrogenated vegetable oils, sodium benzoate, sodium acetate, leucine, PEG-4000 and magnesium lauryl sulfate. Certain additives for use with the present invention, and include: Talc: Talc is a purified, hydrated, magnesium silicate. It is widely used in oral solid dosage forms as a lubricant and diluent.

In some compositions, additives may also include: chelating agents (such as EDTA and EDTA salts); colorants or opaquants (such as titanium dioxide, food dyes, lakes, natural vegetable colorants, iron oxides, silicates, sulfates, magnesium hydroxide and aluminum hydroxide); coolants (e.g., trichloroethane, trichloroethylene, dichloromethane, fluorotrichloromethane); cryoprotectants (such as trehelose, phosphates, citric acid, tartaric acid, gelatin, dextran and mannitol); and diluents or fillers (such as lactose, mannitol, talc, magnesium stearate, sodium chloride, potassium chloride, citric acid, spray-dried lactose, hydrolyzed starches, directly compressible starch, microcrystalline cellulose, cellulosics, sorbitol, sucrose, sucrose-based materials, calcium sulfate, dibasic calcium phosphate and dextrose). Yet other additives may include disintegrants or super disintegrants; hydrogen bonding agents, such as magnesium oxide; flavorants or desensitizers.

It should be appreciated that there is considerable overlap between the above-listed additives and/or active agent in common usage, since a given additive/active agent is often classified differently by different practitioners in the field, or is commonly used for any of several different functions. Thus, the above-listed additives should be taken as merely exemplary, and not limiting, of the types of additives that can be included in compositions of the present invention. The amounts of such additives may be readily determined by one skilled in the art, according to the particular properties desired.

A pelletization process typically involves preparing a molten solution of the composition of the solid carrier or a dispersion of the composition of the solid carrier solubilized or suspended in an aqueous medium, an organic solvent, a supercritical fluid, or a mixture thereof. Such solution or dispersion is then passed through a certain opening to achieve the desired shape, size, and other properties. Similarly, appropriate drying processes may be used to control the level of the residual dispersing medium, if necessary. The processes described above, the combination of the processes, or the modifications of the processes are known in the art. Some of the processes are briefly described herein for reference.

In a broad sense, pellets are very much like granules and bead; the techniques for producing pellets may also produce granules, beads, etc. Pellets, granules or beads are formed with the aid of, e.g., a pelletizer, a spheronizer or an extruder. The pelletizer, spheronizer or extruder is able to form approximately spherical bodies from a mass of finely divided particles continuously, by a rolling or tumbling action on a flat or curved surface with the addition of a liquid.

Pelletizers are generally classified based on the angle of their axis as a horizontal drum or an inclined dish pelletizer. Rotary fluidized granulators may also be used for pelletization. A standard fluidized drier bowl may be replaced with a rotating plate as an air distributor. For granulation, a binder liquid is sprayed from via one or two binary nozzles located axially to the rotational movement of the powder bed. The granulation results in rounding of the granules to approximately spherical pellets. Such balling or agitation techniques are generally influenced by operating conditions, e.g., the bridging/binding liquid requirements, the residence time of the material in the pelletizer, the speed and angle of inclination of the pelletizer, the amount of material fed to the pelletizer and the choice and levels of binder, etc. Those skilled in the art may adjust readily such factors to produce a satisfactory product.

A tablet may be made by compression or molding, optionally with one or more accessory ingredients. Compressed tablets may be prepared by compressing, in a suitable machine, the therapeutic ingredient in a free-flowing form such as a powder or granules, optionally mixed with a binder, lubricant, inert diluent, preservative, surface therapeutic or dispersing agent. Molded tablets may be made by molding, in a suitable machine, a mixture of the powdered compound moistened with an inert liquid diluent. The tablets may be optionally coated or scored and may be formulated so as to provide a slow or controlled release of the therapeutic ingredient therein.

The choice of binder for a given application may also be determined readily by those skilled in the art. Generally, the binder must be capable of wetting the surfaces of the particle being pelletized or granulated. In general, binders must have sufficient wet strength to allow agglomerates to be handled and sufficient dry strength to make them suitable for their intended purposes. Each process, however, makes use of a different system of forces and may require a different agglomerate strength. The final selection of the binder is made generally based on the type of equipment used. Factors that affect the equipment and binder choices include: the size and size distribution of pellets, bulk density, strength and flow properties. Other factors that affect the performance of the pellets, which may be adjusted by one skilled in the art by the inclusion of additives, choice of equipment and processing conditions.

For example, suitable polymers for use with the present invention include, but are not limited to, synthetic polymers such as poly(ethylene glycol), poly(ethylene oxide), partially or fully hydrolyzed poly(vinyl alcohol), poly(vinylpyrrolidone), poly(ethyloxazoline), poly(ethylene oxide)-co-poly(propylene oxide) block copolymers (poloxamers and meroxapols), poloxamines, carboxymethyl cellulose, and hydroxyalkylated celluloses such as hydroxyethyl cellulose and methylhydroxypropyl cellulose, and natural polymers such as polypeptides, polysaccharides or carbohydrates such as Ficoll®, polysucrose, hyaluronic acid, dextran, heparan sulfate, chondroitin sulfate, heparin, or alginate, and proteins such as gelatin, collagen, albumin, or ovalbumin or copolymers or blends thereof. As used herein, “celluloses” includes cellulose and derivatives of the types described above; “dextran” includes dextran and similar derivatives thereof.

The blend of polymers may form a hydrogel or matrix using a material such as a carbohydrate polymer or polysaccharide (e.g., hyaluronic acid) in the presence of an initiator such as mono-, di- or trivalent cations or anions in water, a radical, or a photoinitiator. The polymer blend may be intrinsically biodegradable, biocompatible, or of sufficiently low molecular weight to allow excretion. Some components of the polymer blend exhibit little to no ability to biologically degrade. Where there are two or more water-soluble polymer blocks joined by other groups, the joining groups may include biodegradable linkages, polymerizable linkages, or both.

Other polymer formulations for use with the present invention include scaffolds prepared with the polymer of the present invention and one or more bioactive compounds or active species so that the polymer or scaffold becomes a microcarrier for one or more active species. The active species may be incorporated into the polymer or polymer solution (e.g., scaffold) or may be attached to its surface using techniques readily apparent to those skilled in the art. In some instances, it may be preferred to incorporate or attach a precursor of the active agent, e.g., an inactive version of the species that can then be activated to the active species as needed and required. The active species may be a drug or other biologically active compound; thus, the scaffold may be a microcarrier for the delivery of drugs or other biologically active compounds when used in the body. Examples of biologically active compounds are proteins, peptides, polysaccharides, nucleic acids, oligonucleotides, natural and synthetic organic or inorganic molecules, and those biologic molecules used for therapeutic, prophylactic or diagnostic purposes. Drugs may include antibiotics, antivirals, chemotherapeutic agents, anti-angiogenic agents, hormones, anti-inflammatory agents, drugs having an effect on vascular flow or that are effective against one or more diseases, and combinations thereof.

Active agents (excipients) of the present invention can also include decongestants (along with a salt form). Examples include, but are not limited to, phenylephrine (bitartrate, tannate, HBr, HCl), phenylpropanolamine (HCl) and pseudoephedrine (HCl). Furthermore, a number of herbal and/or natural decongestants are known in the art, all of which can be used with the present invention.

Active agents such as expectorants can also be used with the present invention. e.g., guaifenesin, terpin hydrate, (glyceryl guaiacolate), potassium (iodide, citrate) and potassium guaicolsulfonate. Other expectorants, whether individual ingredients or combinations of ingredients may be used with the present invention. Furthermore, a number of herbal and/or natural expectorants are known in the art, all of which may be used with the present invention, e.g., Oregano Leaf Extract 25-500 mg (which may be a liquid extract), Red Clover 25-500 mg, Buckthorn Root 25-500 mg, or Fenugreek 25-500 mg, or mixtures thereof.

Examples of antihistamines for use as active agents with the present invention (e.g., in salt form) are chlorpheniramine (maleate), brompheniramine (maleate), dexchlorpheniramine (maleate), dexbrompheniramine (maleate), triprolidine (HCl), diphenhydramine (HCl), doxylamine (succinate), tripelennamine (HCl), cyproheptatine (HCl), bromodiphenhydramine (HCl), phenindamine (tartrate), pyrilamine (maleate, tannate) and azatadine (maleate). Antitussives that may be used with the present invention (with salt form) include: caramiphen (edisylate), dextromethorphan (HBr) and codeine (phosphate, sulfate). A number of herbal and/or natural antihistamines are known in the art, all of which may be used with the present invention.

Other actives may also be included with the present invention, e.g., non-steroidal anti-inflammatory drugs (NSAIDs) such as propionic acid derivatives; acetic acid derivatives; fenamic acid derivatives; biphenylcarboxylic acid derivatives; and oxicams. Examples of propionic acid derivatives include: ibuprofen, naproxen, ketoprofen, flurbiprofen, fenoprofen, suprofen, fenbufen, and fluprofen may be mentioned as preferred compounds. Acetic acid derivatives include: tolmetin sodium, zomepirac, sulindac and indomethacin. Fenamic acid derivatives include: mefenamic acid and meclofenamate sodium. Diflunisal and flufenisal are biphenylcarboxylic acid derivatives, while oxicams include piroxicam, sudoxicam and isoxicam. Other analgesics for use with the present invention include acetaminophen and phenacetin.

In some embodiments, the present invention can include pharmaceutical Glaze (e.g., Shellac). Shellac is a natural occurring material, consisting of a complex mixture of constituents. The main component of shellac (˜95%) is a resin that upon mild basic hydrolysis gives a mixture of compounds of high plasticity. Shellac is used extensively in the pharmaceutical industry as a film coating agent for beads and tablets.

The one or more active agents that are formulated in a self-stable manner using the present invention may include a wide variety of uses, not just the traditional pharmaceutical agents. Actives for use with the present invention in immediate and/or controlled release formulations may include systemically active therapeutic agents, locally active therapeutic agents, disinfecting agents, chemical impregnants, cleansing agents, deodorants, fragrances, dyes, animal repellents, insect repellents, fertilizing agents, pesticides, herbicides, fungicides, and plant growth stimulants, and the like. Although some examples of active agents are listed, those skilled in the art will appreciate that any of these compounds may be used in the form of their pharmaceutically acceptable salt forms, e.g., carboxylic acids, with counter-ions, e.g., potassium, sodium, calcium; as ionic combinations with, e.g., resins, polymers, beads, matrices; with sugars or sugar derivatives, e.g., malate, tannate; amino acids, lipids, oils or combinations, mixtures and the like. In some embodiments, the present inventors have found that certain actives may be provided with two different salts, each of which may have a different solubility and/or release profile under, e.g., physiologic conditions.

Some other examples of active agents (ingredients) suitable for use in the pharmaceutical formulations and methods of the present invention include: hydrophilic, lipophilic, amphiphilic or hydrophobic, and that can be solubilized, dispersed, or partially solubilized and dispersed, on or about a carrier. The active agent-carrier combination may be coated further to encapsulate the agent-carrier combination. Alternatively, an active ingredient may also be provided separately from the solid pharmaceutical composition, such as for co-administration. Such active ingredients can be any compound or mixture of compounds having therapeutic or other value when administered to an animal, particularly to a mammal, such as drugs, nutrients, cosmaceuticals, nutraceuticals, diagnostic agents, nutritional agents, and the like. The active agents listed below may be found in their native state, however, they will generally be provided in the form of a salt. The active agents listed below include their isomers, analogs and derivatives.

In one embodiment, the active ingredient agent is hydrophobic. Hydrophobic active ingredients are compounds with little or no water solubility. Intrinsic water solubilities (i.e., water solubility of the unionized form) for hydrophobic active ingredients are less than about 1% by weight, and typically less than about 0.1% or 0.01% by weight. Suitable hydrophobic active ingredients are not limited by therapeutic category, and can be, for example, analgesics, anti-inflammatory agents, antihelmimthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, crectile dysfunction improvement agents, immunosuppressants, anti-protozoal agents, anti-thyroid agents, anxiolytic agents, sedatives, hypnotics, neuroleptics, .beta.-Blockers, cardiac inotropic agents, corticosteroids, diuretics, anti-parkinsonian agents, gastro-intestinal agents, histamine receptor antagonists, keratolytics, lipid regulating agents, anti-anginal agents, cox-2 inhibitors, leukotriene inhibitors, macrolides, muscle relaxants, nutritional agents, opioid analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, and mixtures thereof. Salts, isomers and derivatives of the above-listed hydrophobic active ingredients may also be used, as well as combinations and mixtures thereof.

Other examples of suitable hydrophobic active ingredients include: acetretin, albendazole, albuterol, aminoglutethimide, amiodarone, amlodipine, amphetamine, amphotericin B, atorvastatin, atovaquone, azithromycin, baclofen, beclomethasone, benezepril, benzonatate, betamethasone, bicalutanide, budesonide, bupropion, busulfan, butenafine, calcifediol, calcipotriene, calcitriol, camptothecin, candesartan, capsaicin, carbamezepine, carotenes, celecoxib, cerivastatin, cetirizine, chlorpheniramine, cholecalciferol, cilostazol, cimetidine, cinnarizine, ciprofloxacin, cisapride, clarithromycin, clemastine, clomiphene, clomipramine, clopidogrel, codeine, coenzyme Q10, cyclobenzaprine, cyclosporin, danazol, dantrolene, dexchlorpheniramine, diclofenac, dicoumarol, digoxin, dehydroepiandrosterone, dihydroergotamine, dihydrotachysterol, dirithromycin, donezepil, efavirenz, eposartan, ergocalciferol, ergotamine, essential fatty acid sources, etodolac, etoposide, famotidine, fenofibrate, fentanyl, fexofenadine, finasteride, fluconazole, flurbiprofen, fluvastatin, fosphenyloin, frovatriptan, furazolidone, gabapentin, gemfibrozil, glibenclamide, glipizide, glyburide, glimepiride, griseofulvin, halofantrine, ibuprofen, irbesartan, irinotecan, isosorbide dinitrate, isotretinoin, itraconazole, ivermectin, ketoconazole, ketorolac, lamotrigine, lansoprazole, leflunomide, lisinopril, loperamide, loratadine, lovastatin, L-thryroxine, lutein, lycopene, medroxyprogesterone, mifepristone, mefloquine, megestrol acetate, methadone, methoxsalen, metronidazole, miconazole, midazolam, miglitol, minoxidil, mitoxantrone, montelukast, nabumetone, nalbuphine, naratriptan, nelfinavir, nifedipine, nilsolidipine, nilutanide, nitrofurantoin, nizatidine, omeprazole, oprevelkin, oestradiol, oxaprozin, paclitaxel, paracalcitol, paroxetine, pentazocine, pioglitazone, pizofetin, pravastatin, prednisolone, probucol, progesterone, pseudoephedrine, pyridostigmine, rabeprazole, raloxifene, rofecoxib, repaglinide, rifabutine, rifapentine, rimexolone, ritanovir, rizatriptan, rosiglitazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafine, terazosin, tetrahydrocannabinol, tiagabine, ticlopidine, tirofibran, tizanidine, topiramate, topotecan, toremifene, tramadol, tretinoin, troglitazone, trovafloxacin, ubidecarenone, valsartan, venlafaxine, verteporfin, vigabatrin, vitamin A, vitamin D, vitamin E, vitamin K, zafirlukast, zileuton, zolmitriptan, zolpidem, and zopiclone. Of course, salts, isomers and derivatives of the above-listed hydrophobic active ingredients may also be used, as well combinations and mixtures thereof.

In other embodiments, the active ingredient is hydrophilic, however, combination of hydrophilic, hydrophobic and non-polar agents may also be used. The water solubility for hydrophilic active ingredients is generally greater than about 0.1% by weight, and typically greater than about 1% by weight. Suitable hydrophilic active ingredients include: analgesics, anti-inflammatory agents, antihelminthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, .beta.-Blockers, cardiac inotropic agents, corticosteroids, diuretics, anti-parkinsonian agents, gastro-intestinal agents, histamine receptor antagonists, keratolytics, lipid regulating agents, anti-anginal agents, cox-2 inhibitors, leukotriene inhibitors, macrolides, muscle relaxants, nutritional agents, opioid analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, and mixtures thereof.

Other hydrophilic active ingredients include: a cytokine, a peptidomimetic, a peptide, a protein, a toxoid, a serum, an antibody, a vaccine, a nucleoside, a nucleotide, a portion of genetic material, a nucleic acid, or a mixture thereof. Other examples of suitable hydrophilic active ingredients include: acarbose; acyclovir; acetyl cysteine; acetylcholine chloride; alatrofloxacin; alendronate; aglucerase; amantadine hydrochloride; ambenomium; amifostine; amiloride hydrochloride; aminocaproic acid; amphotericin B; antihemophilic factor (human), antihemophilic factor (porcine); antihemophilic factor (recombinant), aprotinin; asparaginase; atenolol; atracurium besylate; atropine; azithromycin; aztreonam; BCG vaccine; bacitracin; becalermin; belladona; bepridil hydrochloride; bleomnycin sulfate; calcitonin human; calcitonin salmon; carboplatin; carbohydrate and carbohydrate polymers, capecitabine; capreomycin sulfate; cefamandole nafate; cefazolin sodium; cefepime hydrochloride; cefixime; cefonicid sodium; cefoperazone; cefotetan disodium; cefotaxime; cefoxitin sodium; ceftizoxime; ceftriaxone; cefuroxime axetil; cephalexin; cephapirin sodium; cholera vaccine; chorionic gonadotropin; cidofovir; cisplatin; cladribine; clidinium bromide; clindamycin and clindamycin derivatives; ciprofloxacin; clodronate; colistimethate sodium; colistin sulfate; corticotropin; cosyntropin; cromolyn sodium; cytarabine; dalteparin sodium; danaparoid; desferrioxamine; denileukin diflitox; desmopressin; diatrizoate meglumine and diatrizoate sodium; dicyclomine; didanosine; dirithromycin; dopamine hydrochloride; domase alpha; doxacurium chloride; doxorubicin; etidronate disodium; enalaprilat; enkephalin; enoxaparin; enoxaprin sodium; ephedrine; epinephrine; epoetin alpha; erythromycin; esmolol hydrochloride; factor IX; famciclovir; fludarabine; fluoxetine; foscamet sodium; ganciclovir; granulocyte colony stimulating factor, granulocyte-macrophage stimulating factor; growth hormones—recombinant human; growth hormone—bovine; gentamycin; glucagon; glycopyrolate; gonadotropin releasing hormone and synthetic analogs thereof, GnRH; gonadorelin; grepafloxacin; haemophilus B conjugate vaccine; Hepatitis A virus vaccine inactivated; Hepatitis B virus vaccine inactivated; heparin sodium; indinavir sulfate; influenza virus vaccine; interleukin-2; interleukin-3; insulin-human, insulin lispro; insulin procine; insulin NPH; insulin aspart; insulin glargine; insulin detemir; interferon alpha; interferon beta; ipratropium bromide; ifosfamide; Japanese encephalitis virus vaccine; lamivudine; leucovorin calcium; leuprolide acetate, levofloxacin; lincomycin and lincomycin derivatives; lobucavir; lomefloxacin; loracarbef; mannitol; is measles virus vaccine; meningococcal vaccine; menotropins; mepenzolate bromide; mesalamine; methenamine; methotrexate; methscopolamine; metformin hydrochloride; metoprolol; mezocillin sodium; mivacurium chloride; mumps viral vaccine; nedocromil sodium; neostigmine bromide; neostigmine methyl sulfate; neurontin; norfloxacin; octreotide acetate; ofloxacin; olpadronate; oxytocin; pamidronate disodium; pancuronium bromide; paroxetine; perfloxacin; pentamidine isethionate; pentostatin; pentoxifylline; periciclovir; pentagastrin; pentholamine mesylate; phenylalanine; physostigmine salicylate; plague vaccine; piperacillin sodium; platelet derived growth factor-human; pneumococcal vaccine polyvalent; poliovirus vaccine inactivated; poliovirus vaccine live (OPV); polymyxin B sulfate; pralidoxime chloride; pramlintide, pregabalin; propafenone; propenthaline bromide; pyridostigmine bromide; rabies vaccine; residronate; ribavarin; rimantadine hydrochloride; rotavirus vaccine; salmeterol xinafoate; sinealide; small pox vaccine; solatol; somatostatin; sparfloxacin; spectinomycin; stavudine; streptokinase; streptozocin; suxamethonium chloride; tacrine hydrochloride; terbutaline sulfate; thiopeta; ticarcillin; tiludronate; timolol; tissue type plasminogen activator; TNFR:Fc; TNK-tPA; trandolapril; trimetrexate gluconate; trospectinomycin; trovafloxacin; tubocurarine chloride; tumor necrosis factor; typhoid vaccine live; urea; urokinase; vancomycin; valacyclovir; valsartan; varicella virus vaccine live; vasopressin and vasopressin derivatives; vecuronium bromide; vinblastine; vincristine; vinorelbine; vitamin B12; warfarin sodium; yellow fever vaccine; zalcitabine; zanamivir; zolendronate; zidovudine; pharmaceutically acceptable salts, isomers and derivatives thereof, and mixtures thereof.

A wide variety of therapeutically active agents can be used in conjunction with the present invention. The therapeutically active agents (e.g. pharmaceutical agents) which may be used in the compositions of the present invention include both water soluble and water insoluble drugs. Examples of such therapeutically active agents include antihistamines (e.g., dimenhydrinate, diphenhydramine, chlorpheniramine and dexchlorpheniramine maleate), analgesics (e.g., aspirin, codeine, morphine, dihydromorphone, oxycodone, etc.), non-steroidal anti-inflammatory agents (e.g., naproxyn, diclofenac, indomethacin, ibuprofen, sulindac), anti-emetics (e.g., metoclopramide), anti-epileptics (e.g., phenyloin, meprobamate and nitrezepam), vasodilators (e.g., nifedipine, papaverine, diltiazem and nicardirine), anti-tussive agents and expectorants (e.g., codeine phosphate), anti-asthmatics (e.g. theophylline), antacids, anti-spasmodics (e.g., atropine, scopolamine), antidiabetics (e.g., insulin), diuretics (e.g., ethacrynic acid, bendrofluazide), anti-thypotensives (e.g., propranolol, clonidine), antihypertensives (e.g, clonidine, methyldopa), bronchodilators (e.g., albuterol), steroids (e.g., hydrocortisone, triamcinolone, prednisone), antibiotics (e.g., tetracycline), antihemorrhoidals, hypnotics, psycho-tropics, antidiarrheals, mucolytics, sedatives, decongestants, laxatives, vitamins, stimulants (including appetite suppressants such as phenylpropanolamine), as well as salts, hydrates, and solvates of the same. The above list is not meant to be exclusive.

In certain embodiments, the therapeutically active agent comprises hydromorphone, oxycodone, dihydrocodeine, codeine, dihydromorphine, morphine, buprenorphine, salts, hydrates and solvates of any of the foregoing, mixtures of any of the foregoing, and the like. In other embodiments, the active agent is a locally active therapeutic agent and the environment of use may be, e.g., the gastrointestinal tract, or body cavities such as the oral cavity, periodontal pockets, surgical wounds, the rectum or vagina. The liquid formulations of the present invention may be provided orally, topically, subcutaneously, intramuscularly, intraperitoneally, intraocularly, intraossealy, nasally, urethrally, mucosally, vaginally, rectally, intradurally, epidurally and the like. The liquid formulation of the present invention may also be provided as a mist, e.g., to the deep lung (alveolarly).

Locally active pharmaceutical agents of use with the present invention include antifungal agents (e.g., amphotericin B, clotrimazole, nystatin, ketoconazole, miconazol, etc.), antibiotic agents (penicillins, cephalosporins, erythromycin, tetracycline, aminoglycosides, etc.), antiviral agents (e.g., acyclovir, idoxuridine, etc.), breath fresheners (e.g., chlorophyll), antitussive agents (e.g., dextromethorphan hydrochloride), anti-cariogenic compounds (e.g., metallic salts of fluoride, sodium monofluorophosphate, stannous fluoride, amine fluorides), analgesic agents (e.g., methylsalicylate, salicylic acid, etc.), local anesthetics (e.g., benzocaine), oral anti-septics (e.g., chlorhexidine and salts thereof, hexylresorcinol, dequalinium chloride, cetylpyridinium chloride), anti-inflammatory agents (e.g., dexamethasone, betamethasone, prednisone, prednisolone, triamcinolone, hydrocortisone, etc.), hormonal agents (oestriol), antiplaque agents (e.g, chlorhexidine and salts thereof, octenidine, and mixtures of thymol, menthol, methysalicylate, eucalyptol), acidity reducing agents (e.g., buffering agents such as potassium phosphate dibasic, calcium carbonate, sodium bicarbonate, sodium and potassium hydroxide, etc.), and tooth desensitizers (e.g., potassium nitrate). This list is not meant to be exclusive. Other embodiments of the present invention include disinfecting agents, e.g., chlorine compounds such as calcium hypochlorite, and the environment of use is a surrounding body of water, e.g., a recreational pool. The active may be one or more cleansing agents, a germicide, a deodorant, a surfactant, a fragrance, a perfume, a sanitizer, and/or a dye, and the environment of use is an aqueous solution, e.g., a urinal or toilet bowl. Examples of fragrances include: perfume oils, volatile-compounds including esters, ethers aldehydes, alcohols, unsaturated hydrocarbons, terpenes, and other ingredients well known in the art.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A single dose, composition comprising:
a total of 2m−1 layers, wherein m is an integer between 2 and 100;
a core layer comprising one or more active agents and one or more polymers with a mass ratio greater than about 1;
at least one double layered first composition symmetrically positioned on top and bottom of the core, wherein the first composition comprises one or more active agents and one or more polymers with a mass ratio between about 1 to about (n−1)/n, where n is one or greater; and
at least one double layered second composition symmetrically positioned on top and bottom of the core, wherein the second composition comprises one or more active agents and one or more polymers with a mass ratio between about (n−1)/n to about (n−2)/n, where n is one or greater.

2. The composition of claim 1, further comprises at least one double layered third composition symmetrically positioned on top and bottom of the core, wherein the third composition comprises one or more active agents and one or more polymers with a mass ratio between about (n−2)/n to about (n−3)/n; wherein n=m and is greater or equal to three.

3. The composition of claim 1, further comprises at least one double layered fourth composition symmetrically positioned on top and bottom of the core, wherein the fourth composition comprises one or more active agents and one or more polymers with a mass ratio between about (n−3)/n to about (n−4)/n and wherein n=m and is greater or equal to four.

4. The composition of claim 1, further comprises an external lateral coating having one or more substantially less water permeable or impermeable polymer layer covering the sides of the core to prevent dissolution or release of the one or more active agents.

5. The composition of claim 1, further comprises at least one additional double layered composition comprising a mixture of the one or more active agents and the one or more polymers having a mass ratio of about 0.01-1.00 to about 0.01-1.00.

6. The composition of claim 1, further comprises at least one double layered composition comprising the one or more polymers.

7. The composition of claim 1, wherein each of the double layered composition is arranged in any order.

8. The composition of claim 1, wherein the pharmaceutical composition further comprises one or more layers of an extended-release coating.

9. The composition of claim 1, wherein the pharmaceutical composition further comprises one or more inactive agents.

10. The composition of claim 1, wherein the pharmaceutical composition comprise a substantially constant release rate profile or a release profile in a zero order.

11. A method of making a multilayer composition comprising the steps of:
forming a core comprising one or more active agents and one or more polymers with a mass ratio greater than about 1;
forming at least one double layered first composition symmetrically positioned on top and bottom of the core, wherein the first composition comprises one or more active agents and one or more polymers with a mass ratio between about 1 to about (n−1)/n; and
forming at least one double layered second composition symmetrically positioned on top and bottom of the core, wherein the second composition comprises one or more active agents and one or more polymers with a mass ratio between about (n−1)/n to about (n−2)/n, wherein n is greater or equal to two.

12. The method of claim 11, further comprising the steps of forming at least one double layered third composition symmetrically positioned on top and bottom of the core, wherein the third composition comprises one or more active agents and one or more polymers with a mass ratio between about (n−2)/n to about (n−3)/n, and wherein n is greater or equal to three.

13. The method of claim 11, further comprising the steps of forming at least one double layered fourth composition symmetrically positioned on top and bottom of the core, wherein the fourth composition comprises one or more active agents and one or more polymers with a mass ratio between about (n−3)/n to about (n−4)/n, and wherein n is greater or equal to four.

14. The method of claim 11, further comprising the steps of forming at least one addition layer of composition comprising a mixture of the one or more active agents and the one or more polymers having a molar ratio of about 0.01-1.00 to about 0.01-1.00.

15. The method of claim 11, further comprising the steps of forming an external lateral coating having one or more substantially less water permeable or impermeable polymer layer covering the sides of the core to prevent dissolution or release of the one or more active agents.

16. The method of claim 11, further comprising the steps of forming at least one double layered composition comprising the one or more polymers.

17. The method of claim 11, further comprising the steps of arranging each of the double layered composition in any order.

18. The method of claim 11, further comprising the steps of forming one or more layers of an extended-release coating on the pharmaceutical composition.

19. The method of claim 11, wherein the pharmaceutical composition further comprises one or more inactive agents.

20. The method of claim 11, wherein the pharmaceutical composition comprise a substantially constant release rate profile or a release profile in a zero order.

21. A single dose, pharmaceutical composition comprising:
a core comprising one or more active agents and one or more polymers with a mass ratio greater than about 1;
a first double layered composition symmetrically positioned on top and bottom of the core, wherein the first double layered composition comprises one or more active agents and one or more polymers with a mass ratio between about 1 to about 0.75;
a second double layered composition symmetrically positioned on top and bottom of the core, wherein the second double layered composition comprises one or more active agents and one or more polymers with a mass ratio between about 0.75 to about 0.5;
a third double layered composition symmetrically positioned on top and bottom of the core, wherein the third double layered composition comprises one or more active agents and one or more polymers with a mass ratio between about 0.5 to about 0.25;
a fourth double layered composition symmetrically positioned on top and bottom of the core, wherein the fourth double layered composition comprises one or more active agents and one or more polymers with a mass ratio between about 0.25 to about 0.05; and
an external lateral coating having one or more substantially less water permeable or impermeable polymer layer covering the sides of the core to prevent dissolution or release of the one or more active agents.

22. The composition of claim 21, further comprises at least one additional double layered composition comprising a mixture of the one or more active agents and the one or more polymers having a mass ratio of about 0.01-1.00 to about 0.01-1.00.

23. The composition of claim 21, further comprises at least one double layered composition comprising the one or more polymers.

24. The composition of claim 21, wherein each of the double layered composition is arranged in any order.

25. The composition of claim 21, wherein the pharmaceutical composition further comprises one or more layers of an extended-release coating.

26. The composition of claim 21, wherein the pharmaceutical composition further comprises one or more inactive agents.

27. The composition of claim 21, wherein the layers have at least one closed face that is impermeable.
